(19) United States
(12) Patent Application Publication
(10) Pub. No.: US 2005/0079616 A1
(43) Pub. Date: Apr. 14, 2005

(54) Method of transducing ES cells

(75) Inventor: Benjamin Eithan Reubinoff, Mevaseret-Zion (IL)

Correspondence Address: SCULLY SCOTT MURPHY & PRESSER, PC, 400 GARDEN CITY PLAZA, GARDEN CITY, NY, 11530, US

(73) Assignee: ES Cell International PTE Ltd., Prahran (AU)

(21) Appl. No.: 10/876,071

(22) Filed: Jun. 24, 2004

Related U.S. Application Data
(63) Continuation of application No. PCT/AU02/01758, filed on Dec. 24, 2002.

(30) Foreign Application Priority Data
Dec. 24, 2001 (AU) PR9735
Jun. 4, 2002 (AU) PS2777

(51) Int. Cl. C12N 15/867; C12N 5/08

(52) U.S. Cl. 435/456; 435/366

(57) ABSTRACT

The present invention provides vectors and methods for transducing human embryonic stem cells. Also provided are cells that have been genetically altered using the vectors and methods as well as a protein produced by a transduced cell. Methods for treating an animal having a deficiency in protein are also provided.

### FIELD OF THE INVENTION

The present invention relates to methods for genetically altering embryonic stem cells. The present invention also relates to cells and animals that have been genetically altered using lentiviral vectors.

### BACKGROUND OF THE INVENTION

The ability to introduce genetic modifications into cells via the genetic manipulation of embryonic stem (ES) cells marks a major step in the basic research of the function of many genes of interest. A large proportion of research in this area has been directed to the genetic manipulation of mouse ES cells, since genetically manipulated mouse ES cells are used to alter the mouse genome and thus offer a direct approach to the understanding of gene function in the intact animal. Genetic manipulation of human ES cells may be also invaluable for the study of gene function. It may be also useful for many other applications such as the development of models of diseases, generation of specific ES cell derived progeny, the use of human ES cells in gene therapy and others.

While mouse ES cells are amenable to genetic manipulation by a few approaches, it is known in the art that the biology of murine and human ES cells are quite different in many respects. Accordingly, methods that are efficient with mouse ES cells may be unsuitable for human pluripotent cells.

For example, the cytokine leukemia inhibitory factor (LIF) can support undifferentiated proliferation of mouse ES cells while it has no effect on human ES cells. The differences between human and mouse ES cells were further noted in attempts to provide delivery systems for the transfer and expression of foreign DNA into ES cells using both viral and non-viral vectors. Non-viral gene delivery systems include electroporation, microinjection and cationic liposomes. These systems have a relatively low transfection efficiency and generally allow only transient expression of the transgene. While electroporation is the method of choice for the transfection of mouse ES cells, it has been reported to be inefficient with human ES cells.

In contrast to non-viral delivery systems, viral vectors have proved to be a more efficient tool for gene transfer. Retroviral vectors, which infect only dividing cells, have been used for gene delivery. These vectors integrate into the host DNA and thus enable a stable transgene expression. However, such vectors have problems when used with ES cells due to complete or partial silencing of the transgene in mouse ES and EC cells. This gene silencing is thought to be mediated by de novo methylation of the integrated virus, and by trans-acting transcriptional repressors.

HIV-based lentiviral vectors that have been developed recently are a subgroup of retroviruses. Like retroviruses, the lentivirus vectors integrate into the host DNA. While they infect dividing cells, they can also infect nondividing cells. Lentiviral vectors have been shown to transduce various types of cells including neurons and hepatocytes, in vivo and in vitro.

A LIF dependent feeder free culture system, which is not available for human ES cells, was used to transduce mouse ES cells with the VSV-G lentiviral vector. Results from the Applicant's laboratory have indicated that the efficiency of genetic modification of human ES cells with a VSV-G lentiviral vector similar to the one that was used for the mouse system was low.

Lentiviral vectors have a further problem with regard to biosafety. There is a danger that the introduction of these vectors into an animal may result in the reconstitution of productive viral particles inside a recipient cell. In order to improve the biosafety of the lentiviral vectors several modifications have been introduced into them. First, six viral genes encoding probable virulence factors were deleted from the wild-type virus. Second, a three-plasmid vector system was constructed, separating the remaining genes into three different plasmids to prevent reconstitution of productive viral particles. Third, a self-inactivating (SIN) HIV-1 vector with a deletion in the viral LTR that abolishes its promoter activity has been constructed. The SIN vector can transduce neurons in vivo as efficiently as a vector with full-length LTRs. Its efficiency with ES cells and its potential to induce stable non-silenced expression of transgenes in ES cells is unknown.

To date, the prior art has not disclosed an efficient method for genetically modifying a human ES cell using a lentivirus vector. The prior has also failed to disclose a method for efficiently genetically modifying a feeder layer that supports the undifferentiated propagation of human ES cells. It is therefore an object of the invention to overcome or alleviate a problem of the prior art by providing methods for the stable genetic modification of ES cells using lentiviral vectors.

### SUMMARY OF THE INVENTION

In one aspect the present invention provides a method for transducing a human embryonic stem cell, said method including exposing the embryonic stem cell to a lentiviral vector.

In another aspect the present invention provides a method for treating an animal having a deficiency in a protein, the method including the steps of:
transducing a human ES cell according to the methods described herein, engrafting the cell to the animal, and
allowing the cell to express the protein.

In another aspect the present invention provides an ES cell that has been genetically modified by a method as described herein.

In another aspect the present invention provides a protein produced by a cell genetically modified by a method as described herein.

### DESCRIPTION OF THE FIGURES

FIG. 1 shows a schematic representation of the lentiviral vector construct pRLL.cPPT.hEF-1 αp.eGFP.WPRE.SIN-18.

FIG. 2 shows FACS analysis of transduction of hES cells with 10× concentrated recombinant viruses. Transduced cells were analysed 7 days after transduction for fluorescence intensity and compared to control cells. The level of spontaneous differentiation among the transduced and control human ES cells was determined by the analysis of the proportion of cells that were immunoreactive with the monoclonal antibody GCTM2 (an antibody that reacts with the TRA-1-60 ES cell-surface marker). The upper right and the lower right quadrants represent the nondifferentiated and differentiated transduced hES cells. (A) Non transduced hES cells; (B) hES cells transduced with pRLL.hEF-1αp.eGFP.WPRE.SIN-18; (C) hES cells transduced with pRLL.cPPT.hEF-1αp.eGFP.WPRE.SIN-18.

FIG. 3 shows FACS analysis of human ES cells 45 days (5 passages) after transduction with the lentiviral vector pRLL.cPPT.hEF-1αp.eGFP.WPRE.SIN-18. FACS analysis and determination of the level of spontaneous differentiation were performed as described in FIG. 2.

FIG. 4 shows a human ES cell colony 6 days after passage. The cells were transduced 4 weeks earlier.

(scale bar 100 μm).

FIG. 5 shows an intense expression of the transgene within an embryoid body 3 weeks and 5 days after its formation (scale bar 100 m).

FIG. 6 shows phase contrast appearance and fluorescence microscopy analysis of eGFP expression in a neural progenitor sphere derived from hES cells transduced with pRLL.cPPT.hEF-1αp.eGFP.WPRE.SIN-18. A sphere cultured in serum free medium supplemented with bFGF anf EGF(A, B) and 9 days after plating onto an adhesive surface and culture in the absence of growth factors (C, D). Differentiating cells that are emanating from the sphere (C) are expressing the transgene (D). Scale bar 100 mM.

FIG. 7 shows coexpression of the transgene and neural markers demonstrated by indirect immunofluorescence staining. Neural progenitors, 12 hours after disaggregating of spheres and plating on adhesive substrate coexpressing eGFP and nestin (raw A), N-CAM (raw B), and A2B5 (raw C). Cells which displayed the neuronal marker b-tubulin III and coexpressed eGFP were demonstrated three days after plating of neural progenitors on adhesive substrate and culture in the absence of growth factors (raw D).

The left column of images shows phase contrast micrographs of the cells. The next columns show green and red fluorescence images of the cells respectively. Overlay of the green and red fluorescence images is demonstrated in the right column.

### DETAILED DESCRIPTION OF THE INVENTION

In one aspect the present invention provides a method for transducing a human embryonic stem cell, said method including exposing the embryonic stem cell to a lentiviral vector.

The methods described herein have been discovered to be powerful and efficient tools useful in the transduction of human ES cells.

While the use of lentiviral vectors is known in the art for the transduction of cells, successful transducing of human ES cells by using these vectors was not reported. As used herein the term “transducing” refers to the process of genetically altering a host cell using a non-replicative virus-based vector system. Stem cells of all species have a unique biology, being very different from other cells in the body. Stem cells are undifferentiated cells which can give rise to a succession of mature functional cells. For example, a hematopoietic stem cell may give rise to any of the different types of terminally differentiated blood cells. A special class of stem cell is the embryonic stem (ES) cell. As used herein the term “embryonic stem cell” refers to any cell derived from an embryo and being pluripotent, thus possessing the capability of developing into any cell type of the body, an organ, or tissue.

The unique biology of these primitive cells often prevents the direct application of standard transduction and transfection techniques that have proven effective in the genetic modification of differentiated cells.

There is the further complication that human ES cells are quite different to ES cells of other species. Although the study of mouse ES cells provides clues to understanding the differentiation of general mammalian tissues, dramatic differences in primate and mouse development of specific lineages limits the usefulness of mouse ES cells as a model of human development. Mouse and primate embryos differ substantially in the timing of expression of the embryonic genome, in the formation of an egg cylinder versus an embryonic disc, in the proposed derivation of some early lineages, and in the structure and function in the extra-embryonic membranes and placenta.

In addition, growth factor requirements to prevent differentiation are different for human ES cells compared with the requirements for mouse ES cells. In culturing mouse ES cells, support from the feeder layer can be replaced by supplementation with LIF which can prevent differentiation and promotes continuous proliferation. Large concentrations of cloned LIF fail to prevent differentiation of human ES cell lines, and so embryonic fibroblast feeder layers are required to maintain the pluripotency and continuous proliferation of these cells. The use of feeder cells is known to cause problems with the transduction of the stem cells that are cultured with them.

Again, the unique characteristics of human ES cells manifests as a difficulty in transducing these cells. As evidence of this, the work of Hamaguchi et al (2000) demonstrates the stable transduction of mouse ES cells using a VSVG pseudotyped lentiviral vector. However, Applicants have shown that a similar vector is inefficient in the transduction of human ES cells.

Thus, differences between human and non-human stem cells coupled with the further differences between embryonic stem cells and differentiated cells leads to uncertainty when attempting to genetically modify a human ES cell by transduction.

The ability to genetically modify an ES cell is desirable because the genetic modification can potentially be passed to all progeny cells. Thus it is possible to genetically modify every cell of an organism that was derived from a single altered stem cell.

Lentiviral vectors are known in the art for their ability to transduce some cell types and have the advantage of being able to produce a stable insertion of a gene into the host cell genome. Once integrated into the host genome the provirus behaves like a resident gene, and expresses a foreign protein inserted into the viral vector. Lentiviral vectors are superior to other retroviral vectors since they do not have an absolute requirement that at least one cell division occurs in the 24 hours after viral absorption. Human (HIV) and animal (FIV, SIV) lentivirus vectors do not have this limitation. However the genomes of these viruses are complex and some products (e.g. the protease) tend to be toxic to host cells. Thus, while lentiviral vector systems have been a great advance in transducing cells, there is a high level of unpredictability that such vectors will be useful for all cell types in a given species, or across all species for a given cell type.

Even in consideration of the problems disclosed in the prior art, Applicants have successfully applied lentiviral vector technology to the transduction of human ES cells.

In a preferred form of the method the vector includes a central polypurine tract (cPPT) or functional equivalent thereof. As used herein the term “central polypurine tract or functional equivalent thereof” means any DNA sequence that is required in cis for the nuclear import of the viral genome into the nucleus of the host cell. An example of such a sequence is the 99 base pair cPPT sequence form the lentiviral pol gene disclosed by Zennou (2000).

In a more preferred embodiment of the method, the cPPT is derived from a HIV-1, SIV or FIV pol gene. Yet more preferably the cPPT is derived from a HIV-1 pol gene. Most preferably the cPPT is a sequence as disclosed by Genebank accession number NC_{—}001802 (nucleotides 4303-4480). The introduction of the cPPT element into the vector had a remarkable effect (at least 3 fold increase) on the efficiency of transduction.

Transduction of human ES cells can be further improved by minimising the deleterious effects of feeder fibroblasts on transduction efficiency. In a preferred form of the invention transduction is carried out on ES cell clumps that were transiently plated on low density feeders. More preferably the feeders are present at a density of less than 70,000 cells per cm², most preferably the seeding density is about 10,000 cells per cm². Immediately following transduction the ES cell clumps were transferred and further cultured on feeders that were prepared according methods described in PCT/AU99/00990 and PCT/AU01/00278.

Short transduction of 3 hours was found by the Applicants to be as efficient as overnight transduction. Immediately following a short period of transduction the ES cell clumps were transferred and further cultured on feeders that were prepared according methods described in PCT/AU99/00990 and PCT/AU01/00278.

Transduction efficiencies can be further improved by carrying out transduction for short periods on ES cell clumps without the support of feeder layers. In a preferred embodiment of the invention transduction is carried out over about 3 hours. More preferably, double transduction is carried out over about 3 hours. An initial transduction of 1.5 hours is followed by an additional 1.5 hours transduction with fresh supernatant containing virus particles. The double transduction approach improved the transduction efficiency by about 50%.

In a further preferred embodiment of the method, the lentiviral vector is used at a titer of approximately 10⁷ TU/mL. More preferably, the lentiviral vector is used at a concentration of approximately 10⁸ TU/mL. Viral vector concentration and the ratio of the total amount of viral infectious units and the total number of ES cell had a significant effect on the efficiency of transduction. When the viral supernatant was concentrated 10 fold to a titer around 2×10⁸, the transduction efficiency increased 2.5 times or more. A two fold increase in the efficiency of transduction was observed when the total amount of viral infectious units was doubled (0.25 ml vs 0.5 ml at a 2.58×10⁸ TU/ml).

In a preferred aspect of the method the cells are plated on fibronectin. It has been previously demonstrated that the efficiency of lentiviral vector transduction of hematopoietic stem cells may be improved by infecting the cells on fibronectin precoated dishes (Moritz et al 1996). Precoating of the plates with 1 μg/cm² or 20 μg/cm² fibronectin increased the transduction efficiency with human ES cells by two fold (8% as compared to 4%). However it also increased the level of differentiation especially with high concentrations of fibronectin. Among cells that expressed the transgene, 30-40% were also immunoreactive with the GCTM2 antibody when fibronectin precoating was used while 50% were GCTM2 positive when fibronectin precoating was not used. A similar effect was observed with Retronectin (The GCTM2 monoclonal antibody detects an epitope on the protein core of a keratan sulphate/chondroitin sulphate pericellular matrix proteoglycan found in human EC cells (Pera et al., 1988).

In a preferred form of the method the vector further includes a post-transcriptional regulatory element or functional equivalent thereof.

As used herein the term “post-transcriptional regulatory element or functional equivalent thereof” means any DNA sequence capable of increasing transgene expression post-transcriptionally either by increasing stability of the mRNA or by facilitating the nuclear export of the mRNA to the cytoplasm. An example is the Woodchuck Hepatitis B virus post-transcriptional regulatory element disclosed by Zuffrey et al (1999).

In a preferred form of the invention the post-transcriptional regulatory element is from Human or Woodchuck Hepatitis B Virus Post-Transcriptional Regulatory Element (WPRE). More preferably the post transcriptional regulatory element is from the Woodchuck Hepatitis B Virus as disclosed in Genebank accession number J04514 nucleotides 1093-1684.

The level of expression of a transgene after transduction was increased by the inclusion of the post-transcriptional regulatory element into the vector. Where the element is WPRE this modification can increase the mean fluorescence intensity of expression with the construct that contained WPRE by two fold compared with an unmodified vector. Hence the benefits of the additional post-transcriptional regulatory element can be easily ascertained.

Preferably, the vector is prepared from a plasmid vector system comprising at least two plasmids. More preferably the vector is derived from a plasmid vector system comprising three plasmids.

In a preferred embodiment, the vector is a self-inactivating (SIN) vector with a deletion in the viral LTR that abolishes its promoter activity, and/or has at least one virulence factor sequence removed in order to improve the biosafety of the vector. More preferably, all virulence factors are removed in order to improve biosafety. Most preferably, the vector is derived from HIV-1. Before the present invention, the efficiency of SIN vectors for the transduction of ES cells and their potential to induce stable non-silenced expression of transgenes was unknown. The Applicants have surprisingly demonstrated the efficient stable and non-silenced transduction of human ES cells with SIN vectors.

In a further preferred embodiment, the vector is HIV-1 based, pseudotyped with the vesicular stomatitis virus G (VSV-G) protein. Pseudotyping of a lentiviral vector with VSV-G offers two advantages: (1) It broadens the host-cell range of the virus since VSV entrance into host cells is not dependant on specific receptors. (2) It allows for concentration of the virus by ultracentrifugation without loss of infectivity because the VSV-G structurally stabilises the virion.

Preferably, the vector further includes a marker. Cells may be genetically modified at any stage of differentiation with reporter or selectable markers so that the markers are carried through to any stage of cultivation. The markers may be used to purify the differentiated or undifferentiated stem cell population at any stage of cultivation. The use of constructs containing marker genes also enables monitoring the fate of the modified cells after further in vitro or in vivo manipulations.

The vector may further include a selection or reporter marker gene under the control of a cell type specific promoter. Such a construct will be expressed only in a specific cell type and will allow genetic selection for the specific cell and the establishment of a pure population of a specific type of cell. Any cell type may be selected by using this approach including undifferentiated cells or differentiated cells of a specific cell type.

In an even further preferred embodiment of the method the vector includes a foreign gene. As used herein, the term “foreign gene” means any gene that is incorporated into the vector, and which is required for the desired genetic modification of the host cell. The gene used to genetically modify the ES cell may encode any protein. In a preferred embodiment the gene encodes a protein that prevents the differentiation of an ES cell such as the pem gene in the mouse ES cell system. Genetic modification using the pem gene may also facilitate the development of purified undifferentiated ES cell populations through the introduction of vectors expressing a gene encoding a protein that will prevent differentiation.

Human ES cells modified by vectors having genes that direct differentiation as well as reporter or selectable marker genes may have improved efficiency of derivation of purified populations of a specific cell type. Pure populations of ES derived specific cell type will provide an unlimited supply of cells for transplantation therapy.

The gene may also encode transcriptional or other factors that will direct differentiation of the host cell. The differentiation of human ES cells may be forced towards a specific lineage or cell type by the introduction of vectors encoding such genes. For example the overexpression of human Nurr-1 gene, which encodes a transcription factor of the thyroid hormone/retinoic acid nuclear receptor family, may be used to generate dopaminergic neurons (Wagner et al 1999). Accordingly the vectors of the present invention may comprise a gene encoding a protein that controls the differentiation of the host cell.

The skilled person will understand that for the gene to be expressed, the vector must include a promoter operably linked to the foreign gene. The promoter may be any of a number of sequences well known in the art that is capable of initiating expression of a gene in a mammalian cell. Preferably the promoter may be any strong promoter of a “house-keeping” gene. More preferably the promoter is the human polypeptide chain elongation factor 1α (hEF1-α) promoter or hPGK promoter. Most preferably the promoter has a sequence according to Genebank accession number J04617 or Genebank accession number M11958 nucleotides 1-516.

The mean fluorescence intensity of a cell transduced with a vector containing the gene for green fluorescent protein operably linked to the hEF1α promoter was slightly higher than that of a vector incorporating the hPGK promoter. Furthermore, the transduction efficiency was also slightly higher with the hEF1α promoter.

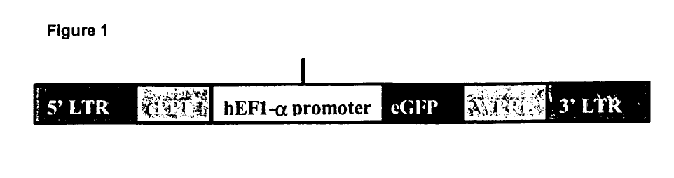

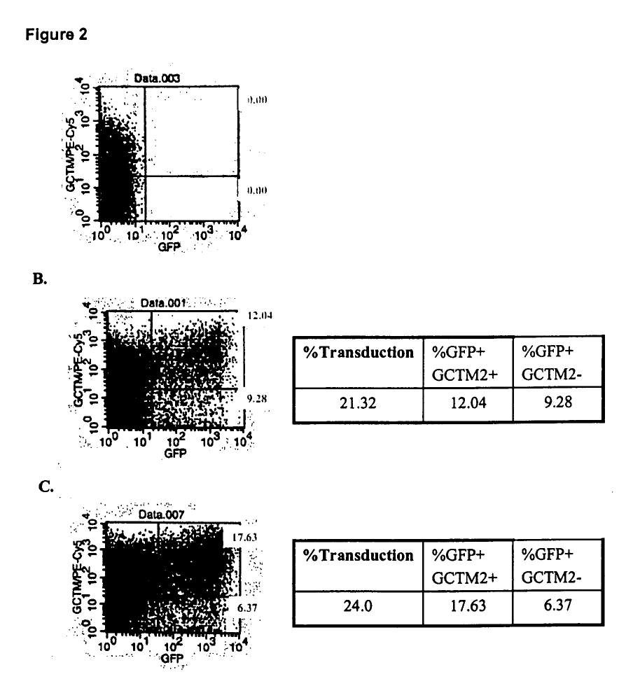

When a concentrated lentiviral vector that included the hEF-1α promoter, WPRE and the cPPT elements (pRLL.cPPT.hEF-1αp.eGFP.WPRE.SIN-18, FIG. 1) was used to transduce human ES cells with a modified protocol as detailed in the Examples section, 24% of the human ES cells expressed the transgene (FIG. 2). Seventy percent of the cells that expressed the transgene were undifferentiated as indicated by immunoreactivity with GCTM2 antibody. Similar proportions of cells were immunoreactive with GCTM2 in control nontransduced cells indicating that the transduction protocol did not induce differentiation.

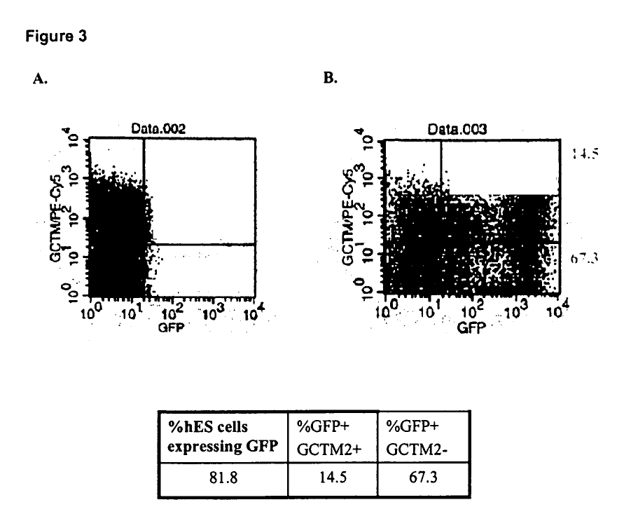

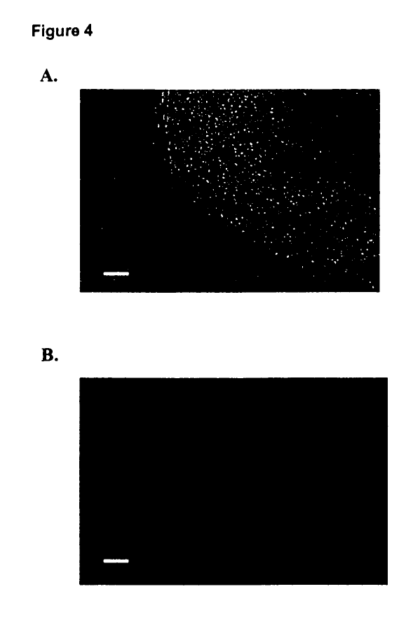

By mechanically passaging regions that expressed eGFP the population of hES expressing eGFP has further increased. Fluorescence microscopy analysis of hES cells 28 days after transduction showed an intense expression of the transgene by the majority of the hES cells (FIG. 4). This was confirmed by FACS analysis of transduced hES cells 45 days after transduction (5 passages) demonstrating that 81% of the hES cells expressed high levels of expression of the transgene (FIG. 3). The transduced cells are now being maintained for 16 weeks without any apparent loss of transgene expression.

Most of the cells were undifferentiated (according to morphological criteria) at the time of transduction. However, 30-60% of the cells were differentiated as indicated by the lack of immunoreactivity with GCTM2 antibody a week later. These cells probably underwent spontaneous differentiation during the week of culture. This level of spontaneous differentiation is common with currently used culture systems. Since the proportion of differentiated cells was similar among cells that expressed or did not express the transgene, it appears that the process of differentiation did not induce silencing of the transgene.

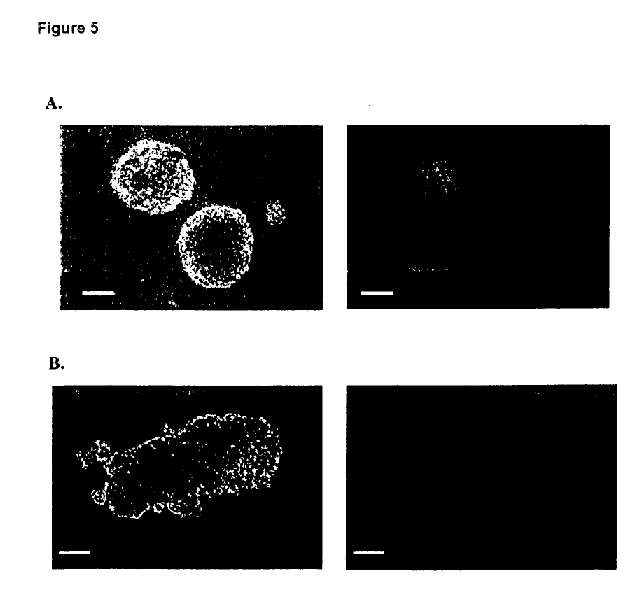

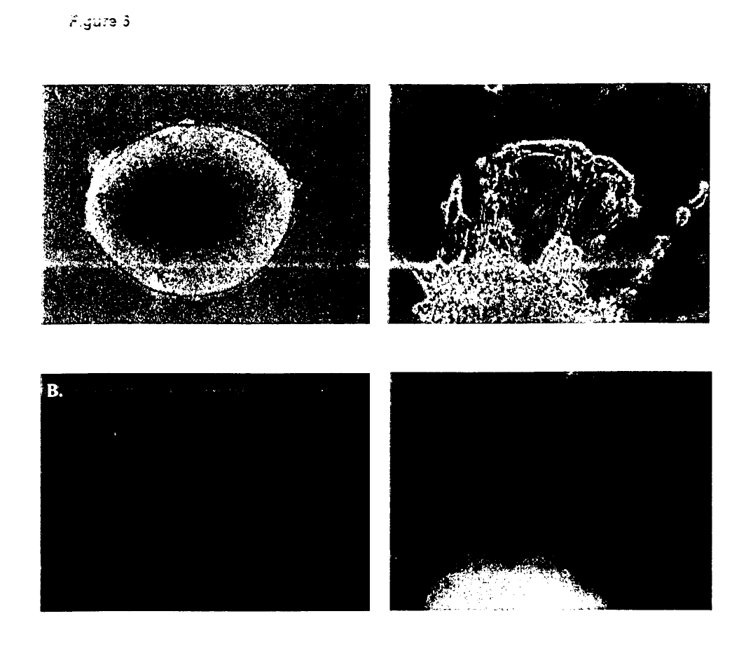

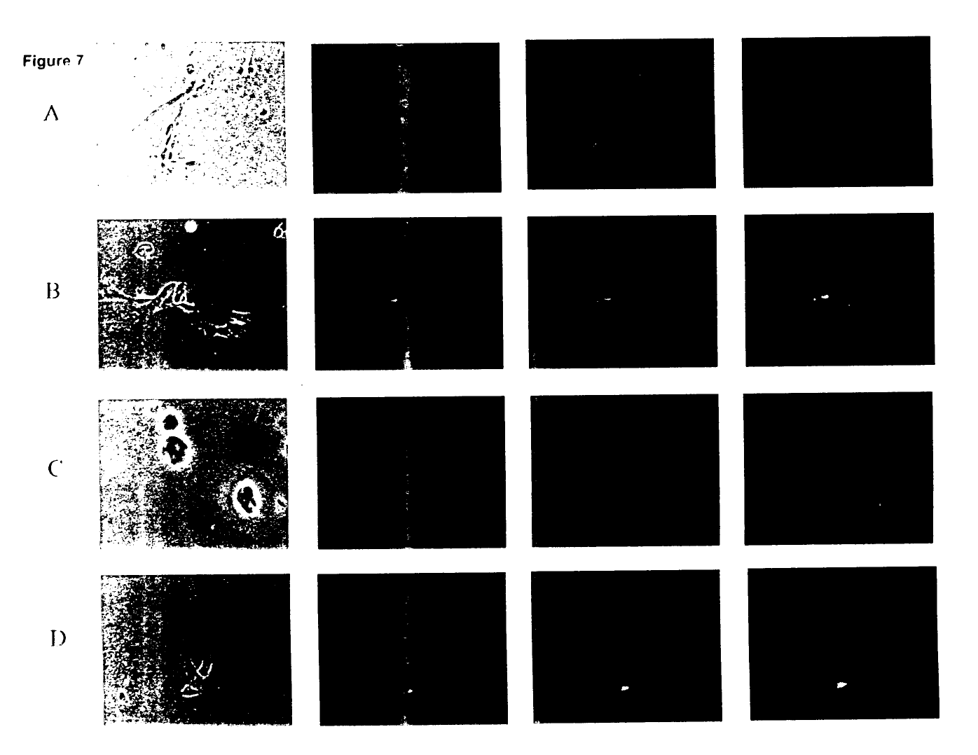

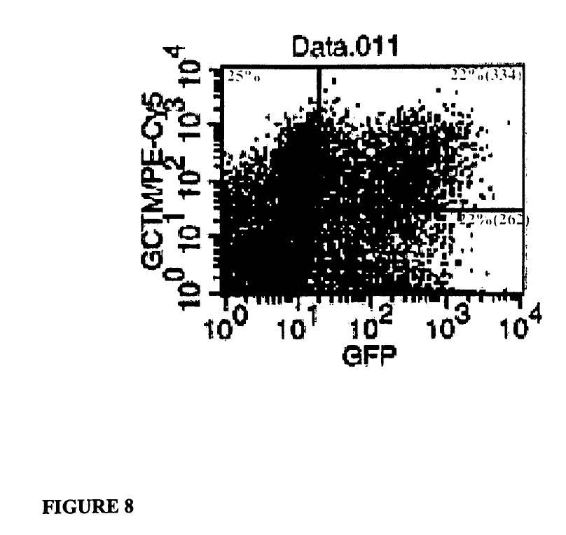

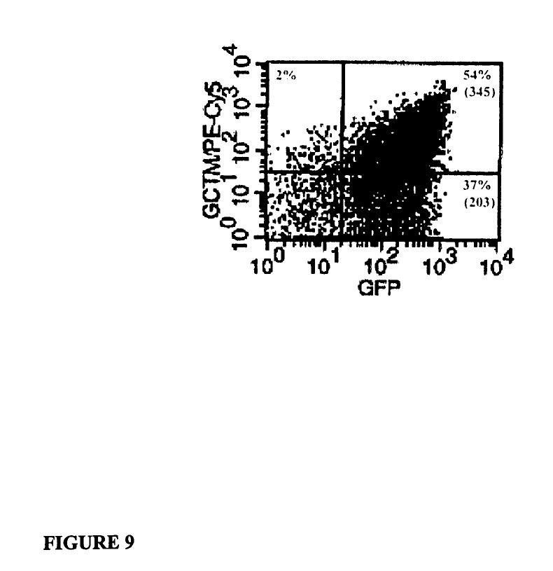

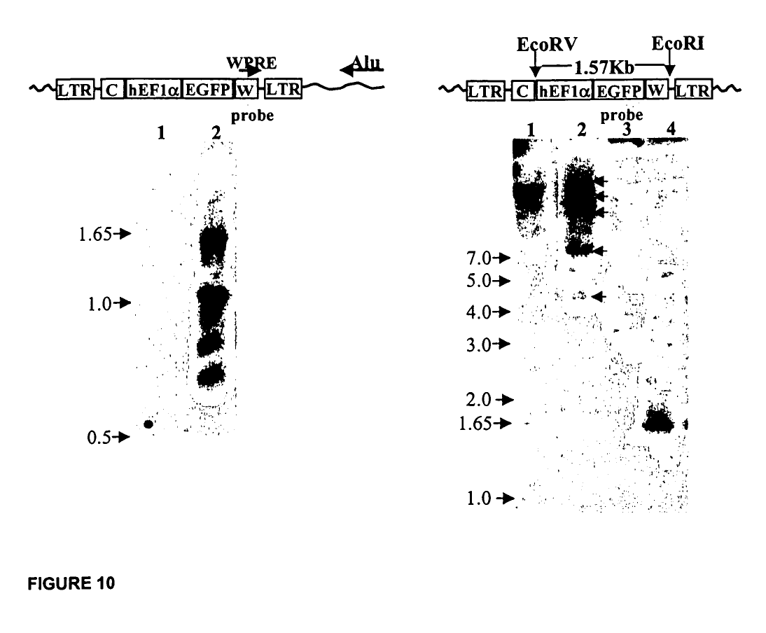

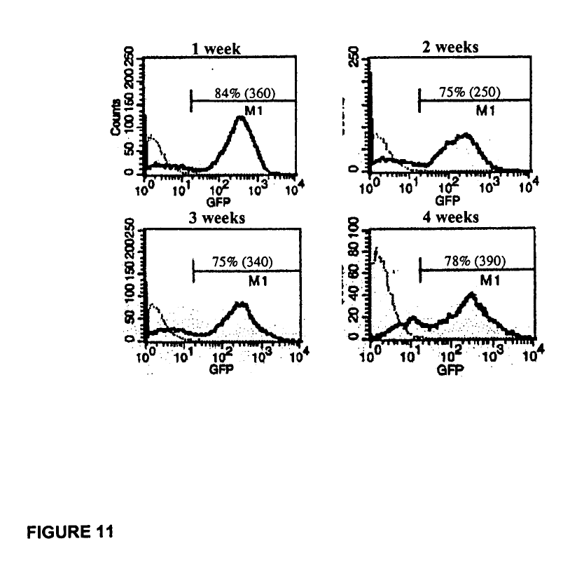

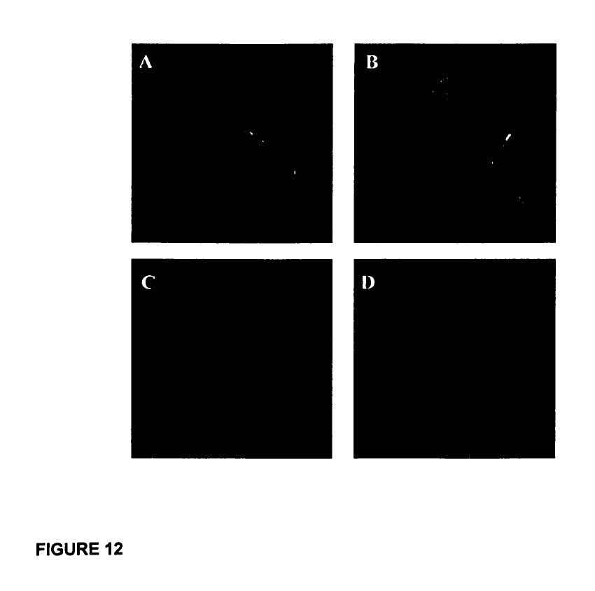

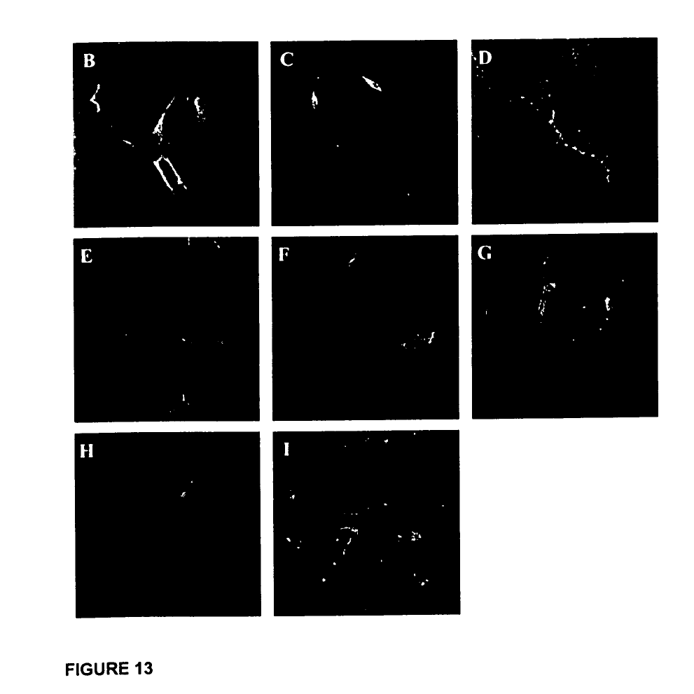

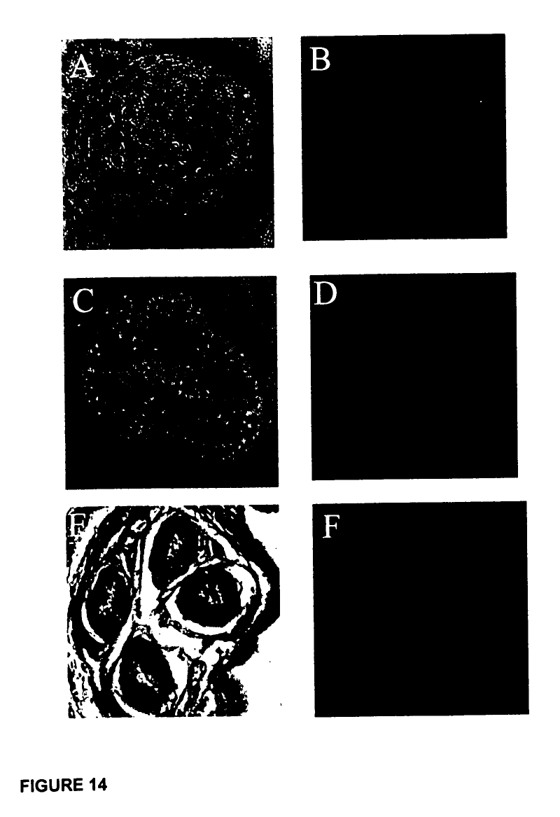

Moreover, 21 days after the generation of embryoid bodies, intense expression of the transgene was documented by fluorescence microscopy (FIG. 5). Transgene expression was also not silenced throughout neural differentiation in vitro. Neural progenitor spheres were derived from transduced hES cells and propagated in culture as previously described (Reubinoff et al 2001). Fluorescence microscopy analysis revealed an intense expression of eGFP within the neural spheres FIG. 6). Cells from 4 week old spheres co-expressed the reporter gene eGFP and markers of primitive neuroectoderm (N-CAM, nestin, vimentin and A2B5) (FIG. 7). Furthermore after induction of differentiation of the neural spheres by plating on an appropriate substrate and removal of mitogens (Reubinoff et al., 2001), the expression of transgene was maintained in differentiating cells (FIG. 6) including neurons as evidenced by the demonstration of eGFP positive cells that displayed the morphology and markers (β-tubulin III and NF-70) of neurons (FIG. 7).

In a preferred form, the method does not induce differentiation of cells.

In another preferred aspect of the present invention the method does not affect the potential of cells to self-renew and proliferate in vitro.

In a further preferred form of the invention, the expression of a transgene included in the vector is not silenced upon replication and/or differentiation of the cell.

Genetic modification may facilitate the development of purified undifferentiated ES cell populations through the use of vectors expressing a selectable marker under the control of a stem cell specific promoter such as Oct-4. The sequence of the Oct-4 promoter may be found in the Genebank database under the accession number Z11900. The Genebank database can be accessed on the Internet at the URL http://www3. ncbi.nlm.nih.gov/Entrez/nucleotide.html.

Other stem cell-specific promoters that can be used are (1) the promoter of the human growth differentiation factor 3 (hGDF3), a member of TGFβ superfamily. The sequence of the region upstream to the transcription start point is available (Genebank accession number AC006927). (2) The promoter of the human transcriptional repressor HFH2 (termed also Genesis), a member of the winged helix transcriptional regulatory family. The human homologue of Genesis was cloned and sequenced (Genebank accession number AF197560).

Some differentiated progeny of ES cells may produce products that are inhibitory to stem cell renewal or survival. Therefore selection against such differentiated cells, facilitated by the introduction of a construct such as that described above, may promote stem cell growth and prevent differentiation.

ES cells may be genetically modified by vectors that may facilitate the elimination of undifferentiated ES cells after transplantation. This may be achieved by transduction with vectors expressing a suicidal marker under the control of a stem cell specific promoter such as Oct-4. Alternatively, expression of marker genes under the control of a stem cell specific promoter that will facilitate specific elimination by immunotherapy or other destructive agents may be used instead of the suicidal gene. Vectors may be also constructed in a similar manner to eliminate specific differentiated cell type both in vitro or in vivo by using lineage/cell type specific promoters.

In another aspect the present invention provides a method for treating an animal having a deficiency in a protein, the method including the steps of:
transducing a human ES cell according to the methods described herein, engrafting the cell to the animal, and
allowing the cell to express the protein.

Cells modified to express a foreign protein can be implanted into an animal where they are able to produce a protein in which the animal is deficient in vivo. The cell may produce the protein before or after engrafting the cell to the animal. The modified ES cells could be used to produce in vivo factors and enzymes that are deficient such as in metabolic disorders such as mucopolysaccharidoses or immune deficiencies such as SCID. Methods for engrafting cells to an animal are known in the art, with some protocols described in (Reubinoff et al 2001).

In another aspect the present invention provides an ES cell that has been genetically modified by a method as described herein. It is contemplated that cells of the present invention may be cultured in vitro to produce a desired protein that may be extracted and purified from the cell culture. Alternatively, the transduced cells may be engrafted into an animal as described above.

In another aspect the present invention provides a protein produced by a cell genetically modified by a method as described herein.

Genetic modification of human ES cells will have a broad range of applications. By genetically manipulating the human ES cells it will be possible to produce either pure stem cell populations or to select for a certain path of differentiation. Pure stem cell populations will be valuable for the analysis of gene expression and function during the early stages of human embryogenesis. Further, they will allow the discovery of new genes associated with pluripotency and early differentiation and to analyze genes involved in developmental failures.

The selection of a certain path of differentiation or a specific type of a differentiated cell may be invaluable for the study of differentiation, the identification of new genes and polypeptide factors which may have a therapeutic potential such as induction of regenerative processes. Additional pharmaceutical applications may include the creation of new assays for toxicology and drug discovery, such as high-throughput screens for neuroprotective compounds. Furthermore, generation of cells of a specific type by genetic modification of human ES cells in vitro may serve as an unlimited source of cells for tissue reconstruction.

Genetic constructs may be inserted to undifferentiated or differentiated cells at any stage of cultivation. The genetically modified cells may be used after transplantation to carry and express genes in target organs in the course of gene therapy.

Genetic modification of ES cells may also be utilized to create in vitro models of diseases. These models may improve understanding of the pathogenesis of various human diseases and serve to develop new therapeutic approaches and drugs.

Genetic modification of ES cells may also be a highly valuable tool for the functional analysis of known and newly discovered genes. Genetic modification using vectors that will over express or modify the expression of genes may allow the determination of a possible role of the trans-acting genes in induction of pluripotency, regulation of stem cell growth, survival and differentiation.

The lentiviral vector system was shown to be a powerful tool for genetic modification of the feeder layer embryonic fibroblasts. In a preferred aspect, the methods described herein may be used to evaluate the effect of over expression or modification of expression of candidate genes in the feeder cells, on the growth, survival and differentiation of ES cells. The methods may be used to express transgenes by the feeders that will improve the potential of the feeders to support undifferentiated proliferation of human ES cells. Alternatively the methods may allow the generation of genetically modified feeders that will direct the differentiation of human ES cells towards a specific cell type. It is contemplated that the present methods may be used to genetically modify feeders to express a selectable gene that is required for genetic selection of feeder dependent human ES cells.

The present invention will now be more fully described with reference to the following examples. It is to be understood that the examples are provided by way of illustration of the invention and that they are in no way limiting to the scope of the invention.

### EXAMPLES

The following materials and methods were used in the following Examples:

Vector Plasmid Construct

The basic transgene construct that was used for transduction of hES cells is pRLL.hPGK.eGFP.SIN-18 (Zufferey et al 1998, Dull et al 1998). This is an HIV-1 based vector containing a large deletion in the 3′ LTR, which abolishes the LTR promoter activity. It expresses eGFP under the transcriptional control of the hPGK promoter. Modifications have been introduced into this vector in order to improve its performance: (1) the hPGK promoter was replaced with the hEF-1α promoter. (2) the post-transcriptional regulatory element from Woodchuck Hepatitis virus-WPRE was inserted downstream to the reporter gene. (3) the central polypurine tract (cPPT) of HIV-1 was reintroduced into the transgene upstream to the hEF-1α promoter. A schematic representation of the final construct, pRLL.cPPT.hEF-1αp.eGFP.WPRE.SIN-18, is presented in FIG. 1:

Vector Production

Vectors were produced by transient cotransfection of three plasmids into 293T cells as described earlier (Naldini et al 1996, Dull et al 1998). Briefly, 1×10⁶ 293T cells were plated on 10 cm plates and transfected the next day using the FuGENE 6 Transfection Reagent (Roche Molecular Biochemicals, Mannheim Germany) with a total of 20 μg of plasmid DNA: 3.5 μg of the envelope plasmid pMD.G, 6.5 μg of the packaging plasmid pCMVΔR8.91, and 1 μg of the transfer vector. The medium was replaced 20-24 h after transfection with the medium for hES cells, but containing 10% fetal bovine serum. The conditioned medium was collected twice, after 24 and 48 hours (48 and 72 hours after transfection) and filtered through 0.45 μm filters (Sartorius, Goettingen Germany).

The control elements that were inserted into the SIN18 vector are as follows: The sequence of the hPGK promoter is found in genebank accession number M11958 nucleotides 1-516. The gene bank accession number for the EF1-αpromoter is J04617. The promoter was amplified by PCR from plasmid pEF-BOS.

The cPPT sequence is found within the complete genome sequence of the HIV-1 (genebank accession number NC_{—}001802 nucleotides 4303-4480. The cPPT fragment was amplified by PCR from a plasmid containing the HIV-1 pol gene (pCMVΔR8.91). The WPRE sequence is found in the full-length sequence of the Woodcuck hepatitis B virus, Genebank accession number J04514 nucleotides 1093-1684. It was amplified by PCR from plasmid HR′-CMV-CXCR4-IRES-GFP-WPRE.

Concentration of Virus

Virus was concentrated by ultracentrifugation in a Sorvall model Discovery 100 centrifuge, in a Surespin 630 swinging bucket rotor, at 50000× g at 4° C. for 1.5 h. After centrifugation the pellet was resuspended at 0.1 of the volume of the original conditioned medium in hES medium (10× concentration). The concentrated virus was used immediately for transduction or stored frozen at −80° C.

Measurement of Viral Titer

293T cells were transduced with serial dilutions of the viral supernatant or the concentrated virus in 12-well plates (1×10⁵ cells/well) in the presence of 5□ g/ml Polybrene. After incubation for 24 h at 37° C. the conditioned medium was removed and fresh medium was added. 4 days after transduction the percentage of the eGFP-positive cells was measured by FACS analysis. The viral titer (Transducing units per milliliter-TU/ml) was calculated by multiplying the percentage of transduced cells by the total number of the cells (1×10⁵) and then dividing it by the volume of the viral supernatant used for transduction.
% transduced cells×total number of cells/volume of the conditioned medium=TU/ml.
   Transduction of ES Cells

Human ES cells (HES-1 cell line) were cultured on mouse embryonic fibroblasts as previously described (Reubinoff et al 2000). Clumps of undifferentiated cells were isolated from 7 days old hES colonies by mechanical slicing followed by dispase digestion (10 mg/ml). 10-14 clumps of the mainly undifferentiated ES cells were incubated with the concentrated virus in the presence of 5 μg/ml Polybrene in 35 mm nontreated tissue culture plates, at 37° C. for 1.5 h. Fresh concentrated virus was then added and the incubation continued for another 1.5 h (“double infection”). In some experiments the plates were precoated with 1 μg/cm² Retronectin (TAKARA Biomedicals, Japan) according to manufacturer instructions. After the incubation the transduced stem cell clumps were collected and replated on mouse feeder layer. Measurement of transduction efficiency was carried out by FACS analysis 7 days after transduction.

Transduction of Mouse embryonic fibroblasts (MEF) was carried out as follows. MEF were treated with mitomycin C and cultured according to our protocol (Reubinoff et al., 2000). The cells were incubated with the concentrated virus in the presence of 5 μg/ml Polybrene at 37° C. over-night.

FACS Analysis

The proportion of cells that expressed the transgene was analyzed on a FACSCalibur system (Becton-Dickenson) according to green fluorescent emission. Nontransduced human ES cells were used to set the background level of fluorescence. Transduced cells were analyzed for fluorescence intensity and compared to control cells. The level of spontaneous differentiation among the transduced and control ES cells was determined by the analysis of the proportion of cells that were immunoreactive with the monoclonal antibody GCTM2 (an antibody that reacts with the TRA-1-60 ES cell-surface marker, Reubinoff et al., 2000).

For FACS analysis the transduced hES colonies were treated with dispase (10 mg/ml), washed with PBS and then trypsinized to obtain a single cell suspension. The cells were centrifuged at 1400 rpm for 4 min and the pellet was incubated with the GCTM-2 antibody on ice for 30 min. The cells were washed with cold hES medium and incubated for further 30 min on ice with PRE-Cy5-Anti-mouse IgG antibody. Then the cells were washed and the pellet was resuspended in FACS buffer.

Immunohistochemistry Studies

Neural spheres were derived from human ES cell colonies and propagated in culture in the presence of bFGF and EGF as previously described (Reubinoff et al 2001). Immunohistochemistry studies were conducted after 4 weeks of propagation. In general, for the immunophenotyping of disaggregated neural progenitor cells and differentiated neurons, fixation with 4% paraformaldehyde for 20 minutes at room temperature was used unless otherwise specified. It was followed by blocking and permeabilization with 0.2% Triton X (Sigma) and 5% heat inactivated goat serum (Dako) in PBS for one hour. Samples were incubated with the primary antibodies at room temperature for 30 minutes, washed, incubated with the secondary antibodies for the same time, counterstained and mounted with Vectashield mounting solution with DAPI (Vector Laboratories, Burlingame, Calif.). Primary antibodies localisation was performed by using mouse anti rabbit IgG and goat anti mouse IgG conjugated to Cy3 (Jackson Lab. West Grove, Pa.: 1:100). Proper controls for primary and secondary antibodies revealed neither non-specific staining nor antibody cross reactivity.

Spheres were disaggregated into single cells that were plated on coverslips that were coated with poly-D-lysine (30-70 kDa, 10 g/ml, Sigma) and laminin (4 μg/ml, Sigma), fixed after 4-12 hours and examined by indirect immunofluorescence analysis for the expression of N-CAM (Dako, Carpinteria, Calif.; 1:10), nestin (rabbit antiserum a kind gift of Dr. Ron McKay; 1:25), A2B5 (ATCC, Manassas, Va.; 1:20) and vimentin (methanol fixation without permeabilization, Roche Diagnostics Australia, Castle Hill, NSW; 1:20). Neuronal differentiation was induced by culturing disaggregated neural progenitors on coverslips coated with poly-D-lysine and laminin in serum free medium without supplementation of growth factors for 3-7 days. Differentiated cells were analysed by indirect immunofluorescence for the expression of the following markers: 70 kDa neurofilament protein (Dako 1:50) and β-tubulin III (Sigma, 1:150).

### Example 1

### Efficient Transduction of Human ES Cells by a Lentiviral Vector

The potential of lentiviral vectors to genetically modify human ES cells was initially evaluated by using the SIN 18 vector (Zufferey et al 1998, Dull et al 1998). The vector included a transgene that was comprised of eGFP under the control of hPGK promoter. Using transient cotransfection of 293 cells with three plasmids we have generated vector titers between 4.3×10⁶ to 3.4×10⁷ TU/ml.

In initial experiments, human ES cell colonies that were cultured on mouse embryonic fibroblasts according to our usual protocol were incubated overnight with conditioned medium containing the viral vector. Intense expression of the transgene was documented within the fibroblasts feeders while sparse expression was observed within the ES cell colonies. To eliminate possible deleterious effect of the fibroblasts on the transduction efficiency we have infected ES cell clumps that were transiently plated on low density feeders (10,000 instead of 70,000 cells per cm²). Immediately following transduction the ES cell clumps were transferred and further cultured on feeders that were prepared according to our standard protocol.

Overnight transduction of hES cells with this protocol resulted in the expression of the transgene by about 2% of the cells. Additional transduction the next morning (double transduction) improved the transduction efficiency by two fold (4%).

To further eliminate possible unwanted effect of the feeders we have developed a protocol where double transduction is conducted for short periods (1.5 hours each) on ES cell clumps that are incubated without the support of feeders. Immediately following the short period of infection (3 hours) the ES cell clumps are further cultured on feeders according to our usual protocol.

It has been previously demonstrated that the efficiency of lentiviral vector transduction of hematopoietic stem cells may be improved by infecting the cells on fibronectin precoated dishes (Moritz et al 1996). Precoating of the plates with 1 μg/cm² or 20 μg/cm² fibronectin increased the transduction efficiency with human ES cells by two fold (8% as compared to 4%). However it also increased the level of differentiation especially with high concentrations of fibronectin. Among cells that expressed the transgene, 30-40% were also immunoreactive with the GCTM2 antibody when fibronectin precoating was used while 50% were GCTM2 positive when fibronectin precoating was not used. A similar effect was observed with Retronectin (The GCTM2 monoclonal antibody detects an epitope on the protein core of a keratan sulphate/chondroitin sulphate pericellular matrix proteoglycan found in human EC cells (Pera et al., 1988).

Viral vector concentration and the ratio of the total amount of viral infectious units and the total number of ES cell had a significant effect on the efficiency of transduction. When the viral supernatant was concentrated 10 fold to a titer around 2×10⁸, the transduction efficiency increased 2.5 times or more. A two fold increase in the efficiency of transduction was observed when the total amount of viral infectious units was doubled (0.25 ml vs 0.5 ml at a 2.58×10⁸ TU/ml).

Concurrently with improving the transduction protocol Applicants have modified the SIN 18 lentiviral vector to improve its performance: (1) the hPGK promoter was replaced with the hEF-1α promoter. (2) the post-transcriptional regulatory element from Woodchuck Hepatitis virus-WPRE was inserted downstream to the reporter gene. (3) the central polypurine tract (cPPT) of HIV-1 was reintroduced into the transgene upstream to the promoter. A schematic representation of the final construct, pRLL.cPPT.hEF-1αp.eGFP.WPRE.SIN-18, is presented in FIG. 1.

Insertion of the WPRE element into the SIN-18 vector did not increase the transduction efficiency, however the mean fluorescence intensity (MFI) of expression with the construct that contained WPRE was two fold higher than the original SIN18 vector.

Replacement of the hPGK promoter with a 208 bp fragment containing the hEF1α promoter slightly increased the MFI of the transgene. Furthermore, the transduction efficiency was also slightly higher.

The introduction of the cPPT element into the vector had a remarkable effect (3 fold increase) on the efficiency of transduction. Moreover the MFI of the transgene was also increased.

When a concentrated SIN18 viral vector that included the hEF-1α promoter, WPRE and the cPPT elements (pRLL.cPPT.hEF-1αp.eGFP.WPRE.SIN-18, FIG. 1) was used to transduce human ES cells with a modified protocol as detailed above, 24% of the human ES cells expressed the transgene (FIG. 2). Seventy percent of the cells that expressed the transgene were undifferentiated as indicated by immunoreactivity with GCTM2 antibody. Similar proportion of cells were immunoreactive with GCTM2 in control nontransduced cells indicating that the transduction protocol did not induce differentiation.

Most of the cells were undifferentiated (according to morphological criteria) at the time of transduction. However, 30-60% of the cells were differentiated as indicated by the lack of immunoreactivity with GCTM2 antibody a week later. These cells probably underwent spontaneous differentiation during the week of culture. This level of spontaneous differentiation is common with our current culture system. Since the proportion of differentiated cells was similar among cells that expressed or did not express the transgene, it appears that the process of differentiation did not induce silencing of the transgene.

### Example 2

### Lentiviral Vectors Promote Stable Expression of the Transgene

The transduced cells were enriched for cells expressing the transgene by selectively passaging cell clumps that expressed the transgene. 28 days after transduction (after 4 selective passages of the cells performed at each consecutive week) an intense expression of the transgene was visualized by fluorescence microscopy (FIG. 4). FACS analysis of the transduced cells 45 days after transduction revealed that 81% of the hES cells maintained high levels of expression of the transgene (FIG. 3). FACS analysis of transduced cells propagated for 12 weeks in culture showed that ˜90% of the hES expressed high levels of the transgene. The transduced cells are now being maintained for 16 weeks without any apparent loss of transgene expression. Thus the transduced hES cells maintain their ability to express the transgene over long periods of cultivation. Moreover the infected undifferentiated cells that expressed the transgene retained the property of self-renewal and proliferation in vitro.

### Example 3

### The Expression of Transgene is not Silenced Upon Differentiation

To examine the effect of differentiation on transgene expression the transduced hES cells were cultivated in suspension to induce their differentiation into embryoid bodies. Fluorscence microscopy analysis of the 5 and 21 days old embryoid bodies revealed intense expression of eGFP within the embryoid bodies (FIG. 5).

Transgene expression was also not silenced throughout neural differentiation in vitro. Neural progenitor spheres were derived from transduced hES cells and propagated in culture as previously described (Reubinoff et al 2001). Fluorescence microscopy analysis revealed an intense expression of eGFP within the neural spheres (FIG. 6). Cells from 4 week old spheres co-expressed the reporter gene eGFP and markers of primitive neuroectoderm (N-CAM, nestin, vimentin and A2B5) (FIG. 7). Furthermore after induction of differentiation of the neural progenitors by plating on an appropriate substrate and removal of mitogens (Reubinoff et al., 2001), the expression of transgene was maintained in differentiating cells (FIG. 6) including the neurons that were generated as evidenced by the demonstration of eGFP positive cells that displayed the morphology and markers (β-tubulin III and NF-70) of neurons (FIG. 7). Thus it seems that transduction of hES cells with the SIN 18 lentiviral vector leads to expression of the transgene that is not silenced upon differentiation.

### Example 4

### Genetic Modification of Feeders by Lentiviral Vector

The lentiviral vector that was used for transduction of hES cells proved efficient for transduction of the mouse feeder layer which supports the growth of the hES cells. Transduction of the mitotically inactivated mouse fibroblasts over-night with a 10× concentrated virus resulted in 30% of fibroblasts expressing the transgene.

### REFERENCES

Barry S C, Harder B, Brzezinski M, Flint L Y, Seppen J, Osborne W R. Lentivirus vectors encoding both central polypurine tract and posttranscriptional regulatory element provide enhanced transduction and transgene expression. Hum Gene Ther 2001; 12(9): 1103-8.
Bartz S R, Vodicka M A. Production of high-titer human immunodeficiency virus type 1 pseudotyped with vesicular stomatitis virus glycoprotein. Methods 1997 12(4):337-42
Blomer U, Naldini L, Kafri T, Trono D, Verma I M, Gage F H. Highly efficient and sustained gene transfer in adult neurons with a lentivirus vector. J Virol 1997; 71(9):6641-9
Cherry S R, Biniszkiewicz D, van Parijs L, Baltimore D, Jaenisch R. Retroviral expression in embryonic stem cells and hematopoietic stem cells. Mol Cell Biol 2000 ;20(20):7419-26.
Dull T, Zufferey R, Kelly M, Mandel R J, Nguyen M, Trono D, Naldini L. J A third-generation lentivirus vector with a conditional packaging system. J Virol 1998; 72(11):8463-71.
Eiges R, Schuldiner M, Drukker M, Yanuka O, Itskovitz-Eldor J, Benvenisty N. Establishment of human embryonic stem cell-transfected clones carrying a marker for undifferentiated cells. Current Biology 2001, 11:514-518.
Fan Y, Melhem M F, Chaillet J R. Forced expression of the homeobox-containing gene Pem blocks differentiation of embryonic stem cells. Dev Biol 1999 ;210(2):481-96.
Follenzi A, Ailles L E, Bakovic S, Geuna M, Naldini L. Gene transfer by lentiviral vectors is limited by nuclear translocation and rescued by HIV-1 pol sequences. Nat Genet 2000;25(2):217-22.
Grez M, Akgun E, Hilberg F, Ostertag. Embryonic stem cell virus, a recombinant murine retrovirus with expression in embryonic stem cells. Proc Natl Acad Sci U S A 1990 ;87(23):9202-6
Hamaguchi I, Woods N B, Panagopoulos I, Andersson E, Mikkola H, Fahlman C, Zufferey R, Carlsson L, Trono D, Karlsson S. Lentivirus vector gene expression during ES cell-derived hematopoietic development in vitro. J Virol 2000 ;74(22): 10778-84.
Jahner D, Stuhlmann H, Stewart CL, Harbers K, Lohler J, Simon I, Jaenisch. De novo methylation and expression of retroviral genomes during mouse embryogenesis. Nature 1982 ;298(5875):623-8
Kafri T, Blomer U, Peterson D A, Gage F H, Verma I M. Sustained expression of genes delivered directly into liver and muscle by lentiviral vectors. Nat Genet 1997 ;17(3):314-7.
Kay M A, Glorioso J C, Naldini L. Viral vectors for gene therapy: the art of turning infectious agents into vehicles of therapeutics. Nat Med 2001 7(1):33-40.
Miyoshi H, Blomer U, Takahashi M, Gage F H, Verma I M. Development of a self-inactivating lentivirus vector .J Virol 1998 Oct;72(10):8150-7.
Moritz T, Dutt P, Xiao X, Carstanjen D, Vik T, Hanenberg H, Williams D A. Fibronectin improves transduction of reconstituting hematopoietic stem cells by retroviral vectors: evidence of direct viral binding to chymotryptic carboxy-terminal fragments. Blood 1996 ;88(3):855-62
Naldini L, Blomer U, Gallay P, Ory D, Mulligan R, Gage F H, Verma I M, Trono D. In vivo gene delivery and stable transduction of nondividing cells by a lentiviral vecto Science 1996 ;272(5259):263-7. Science 1996 ;272(5259):263-7.
Niwa O, Yokota Y, Ishida H, Sugahara T. Independent mechanisms involved in suppression of the Moloney leukemia virus genome during differentiation of murine teratocarcinoma cells. Cell 1983 ;32(4):1105-13
Pera, M. F. et al. Analysis of cell-differentiation lineage in human teratomas using new monoclonal-antibodies to cytostructural antigens of embryonal carcinoma-cells. Differentiation 39, 139-149 (1988).
Reubinoff B E, Pera M F, Fong C Y, Trounson A, Bongso A. Embryonic stem cell lines from human blastocysts: somatic differentiation in vitro. Nat Biotechnol 2000 ;18(4):399-404.
Reubinoff B E, Itsykson P, Turetsky T, Pera M, Reinhartz E, Itzik A, and Ben-Hur T. Neural progenitors from human embryonic stem cells. Nature Biotechnology 2001; 19: 1134-1140
Robertson E J. Using embryonic stem cells to introduce mutations into the mouse germ line. Biol Reprod 1991 ;44(2):238-45.
Stewart C L, Stuhlmann H. Jahner D, Jaenisch R. De novo methylation, expression, and infectivity of retroviral genomes introduced into embryonal carcinoma cells. Proc Natl Acad Sci USA 1982 ;79(13):4098-102.
Vigna E, Naldini L. Lentiviral vectors: excellent tools for experimental gene transfer and promising candidates for gene therapy. J Gene Med 2000 ;2(5):308-16.
Wagner J, Akerud P, Castro D S, Holm P C, Canals J M, Snyder E Y, Perlmann T, Arenas E. Induction of a midbrain dopaminergic phenotype in Nurr1-overexpressing neural stem cells by type 1 astrocytes. Nat Biotechnol 1999 ;17(7):653-9.
Zennou V, Petit C, Guetard D, Nerhbass U, Montagnier L, Charneau P. HIV-1 genome nuclear import is mediated by a central DNA flap. Cell 2000 ;101(2):173-85.
Zufferey R, Donello J E, Trono D, Hope T J Woodchuck hepatitis virus posttranscriptional regulatory element enhances expression of transgenes delivered by retroviral vectors. J Virol 1999 ;73(4):2886-92.
Zufferey R, Dull T, Mandel R J, Bukovsky A, Quiroz D, Naldini L, Trono D. Self-inactivating lentivirus vector for safe and efficient in vivo gene delivery. J Virol 1998 72(12):9873-80.

Finally it is to be understood that various other modifications and/or alterations may be made without departing from the spirit of the present invention as outlined herein.

1. A method for stably transducing a human embryonic stem cell, the method including the steps of
providing a human embryonic stem cell,
exposing the cell to a lentiviral vector capable of integrating into the genome of the cell, the vector including at least one gene operably linked to a promoter, and
maintaining the transduced cell under conditions allowing expression of the gene.

2. A method according to claim 1 wherein the transduced cell is capable of being maintained for at least about 12 weeks without substantial loss of gene expression.

3. A method according to claim 1 wherein the transduced cell is capable of being maintained for at least about 36 weeks without substantial loss of gene expression.

4. A method according to any claim 1 wherein the transduced cell retains pluripotency.

5. A method according to claim 1 wherein expression of the gene is not silenced upon replication and/or differentiation of the transduced cell.

6. A method according to claim 1 wherein the vector includes sequences derived from a virus selected from the group including HIV, FIV and SIV.

7. A method according to claim 1 wherein the vector includes a lentiviral central polypurine tract (cPPT) or functional equivalent thereof.

8. A method according to claim 7 wherein the cPPT is derived from a HIV-1 pol gene.

9. A method according to claim 1 wherein the vector includes a Human or Woodchuck Hepatitis B Virus Post-Transcriptional Regulatory Element (WPRE) or functional equivalent thereof.

10. A method according claim 1 wherein the vector is a self-inactivating (SIN) vector.

11. A method according to claim 1 wherein the vector is HIV-1 based, pseudotyped with the vesicular stomatitis virus G (VSV-G) protein.

12. A method according to claim 1 wherein the gene is a foreign gene.

13. A method according to claim 12 wherein the foreign gene is an antibiotic resistance gene.

14. A method according to claim 1 wherein the gene encodes a protein that prevents the differentiation of the cell.

15. A method according to claim 14 wherein the gene is a pem gene.

16. A method according to claim 1 wherein the gene encodes a transcriptional and/or other factor that directs differentiation of the cell.

17. A method according to claim 1 wherein the promoter is a promoter of a house-keeping gene.

18. A method according to claim 1 wherein the promoter is selected from the group including the human polypeptide chain elongation factor 1α (hEF1-α) promoter, the hPGK promoter, the Oct-4 promoter, the human growth differentiation factor 3 (hGDF3) promoter, and the human transcriptional repressor HFH2 promoter.

19. A lentiviral vector for stably transducing a human embryonic stem cell, the vector including at least one gene operably linked to a promoter.

20. A vector according to claim 19 wherein the vector includes sequences derived from a virus selected from the group including HIV, FIV and SIV.

21. A vector according to claim 19 including a lentiviral central polypurine tract (cPPT) or functional equivalent thereof.

22. A vector according to claim 21 wherein the cPPT is derived from a HIV-1 pol gene.

23. A vector according to claim 19 wherein the vector comprises a Human or Woodchuck Hepatitis B Virus Post-Transcriptional Regulatory Element (WPRE) or functional equivalent thereof.

24. A vector according to claim 19 wherein the vector is a self-inactivating (SIN) vector.

25. A vector according to claim 19 wherein the vector is HIV-1 based, pseudotyped with the vesicular stomatitis virus G (VSV-G) protein.

26. A vector according to claim 19 wherein the gene is a foreign gene.

27. A vector according to claim 19 wherein the gene is an antibiotic resistance gene.

28. A vector according to claim 19 wherein the gene encodes a protein that prevents differentiation of the cell.

29. A vector according to claim 28 wherein the gene is a pem gene.

30. A vector according to claim 19 wherein the gene encodes a transcriptional and/or other factor that directs differentiation of the cell.

31. A vector according to claim 19 wherein the promoter is a promoter of a house-keeping gene.

32. A vector according to claim 19 wherein the promoter is selected from the group including the human polypeptide chain elongation factor 1α (hEF1-α) promoter, the hPGK promoter, the Oct-4 promoter, the human growth differentiation factor 3 (hGDF3) promoter, and the human transcriptional repressor HFH2 promoter.

33. A human embryonic stem cell stably transduced to express a gene product.

34. A population of undifferentiated human embryonic stem cells wherein at least one of the stem cells has been stably transduced to express a gene encoding a protein that inhibits differentiation.

35. A population of undifferentiated human embryonic stem cells according to claim 34 wherein the gene is the pem gene.

36. A human embryonic stem cell transduced by a method according to claim 1 and/or by exposure to a vector according to claim 19.

37. A protein produced by a human embryonic stem cell according to claim 36.

38. A method of treating an animal having a deficiency in a protein, the method including the steps of:
providing a human embryonic stem cell according to claim 36,
engrafting the transduced cell to the animal, and
allowing the cell to express the protein.

39. A method for post transcriptional gene silencing in a human embryonic stem cell, the method including exposing the embryonic stem cell to a vector according to claim 19 wherein the vector includes a nucleotide of silencing the gene.
